(19) United States
(12) Patent Application Publication
(10) Pub. No.: US 2026/0345267 A1
(43) Pub. Date: Nov. 5, 2026

(54) Perioperative Cobot with Tactile and Haptic Feedback for Remote Surgical Interventions

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/432,062

(22) Filed: Dec. 23, 2025

Related U.S. Application Data
(63) Continuation-in-part of application No. 19/195,731, filed on Apr. 30, 2025.

(51) Int. Cl. A61B 34/35; A61B 34/00; A61B 90/00

(52) U.S. Cl. CPC A61B 34/35; A61B 34/25; A61B 34/76; A61B 90/06; A61B 2034/256; A61B 2090/065; A61B 2090/066

(57) ABSTRACT

A perioperative cobotic system enables remote surgical interventions with real-time tactile sensing and haptic feedback. The system includes a robotic platform with dexterous arms capable of sub-millimeter precision, a multi-degree-of-freedom tactile sensor array for detecting force, slip, and tissue compliance, and a haptic feedback module configured to reconstruct touch sensations at a remote control console. A low-latency bi-directional communication architecture ensures real-time transmission of control inputs and sensory data, incorporating buffering and prediction to preserve fidelity. The system includes adaptive feedback modulation, force-limiting algorithms, and safety protocols to prevent excessive force or unintended tissue contact. A surgical console provides immersive control with adjustable force scaling and motion dampening. Real-time procedural logging captures tactile data, control commands, and video for training and compliance. The system supports sterile-field compliance and remote access, enabling precise, safe telesurgical procedures across diverse clinical environments.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to robotic systems for surgical applications and, more particularly, to perioperative cobotic platforms equipped with tactile sensing and haptic feedback technologies for performing or assisting in remote surgical interventions.

### BACKGROUND

The advent of telesurgery has expanded the geographic and logistical reach of expert surgical care by enabling surgeons to operate remotely via robotic systems. Modern telesurgical platforms provide motorized control of robotic instruments and visual feedback through endoscopic or external video streams, allowing surgeons to manipulate tools with increasing dexterity from distant locations. These systems have been instrumental in addressing surgical workforce shortages, supporting military or rural healthcare settings, and enabling collaborative procedures between multiple specialists.

Despite these advancements, current telesurgical systems suffer from a lack of immersive, real-time tactile feedback. Remote surgeons typically rely solely on visual cues and pre-programmed motion constraints, limiting their ability to sense contact forces, texture, compliance, or subtle slip dynamics during tissue manipulation or suturing. The absence of high-fidelity haptic feedback reduces the surgeon's intuitive control and may lead to excessive force application, imprecise dissection, or inadvertent injury to delicate anatomical structures.

Existing robotic platforms also struggle to provide consistent performance under real-world communication constraints. Network latency, jitter, and transient disconnections can compromise control fidelity, delay critical haptic signals, and destabilize the feedback loop between surgeon and robot. Safety mechanisms to prevent excessive force or misalignment are typically limited to static force thresholds or basic collision detection, lacking the contextual awareness needed for complex intraoperative events.

Moreover, conventional systems provide minimal support for procedural documentation, lacking structured capture of tactile interaction data and real-time haptic signals that could inform postoperative review, surgical training, or regulatory compliance. Most telesurgical consoles also lack adaptive interfaces that dynamically adjust haptic response based on the surgical phase, tool type, or patient anatomy, further diminishing clinical precision.

Accordingly, there exists a need for an advanced perioperative cobotic system capable of enabling real-time, high-fidelity tactile and haptic feedback during autonomous, and human controlled telesurgical interventions. Such a system should include precision robotic manipulators equipped with tactile sensor arrays, a haptic generation interface for the remote operator, and a low-latency, bi-directional communication architecture with predictive buffering. In addition, integrated safety compliance logic, adaptive feedback control, and procedural logging capabilities are needed to support safe, responsive, and data-rich remote surgical operations.

### SUMMARY

Embodiments of the present disclosure are directed to a perioperative cobotic system with tactile sensing and haptic feedback for remote surgical interventions. The system enables a surgeon to perform surgical procedures from a remote location with real-time bidirectional control and feedback, closely mimicking the tactile experience of traditional surgery. In general, the system comprises a patient-side cobotic manipulation platform and a surgeon-side immersive control console in communication with each other. The cobotic manipulation platform includes one or more dexterous robotic arms capable of fine motor instrument handling, suturing, and delicate tissue manipulation with sub-millimeter precision. These robotic arms may be operated in a teleoperated mode under the remote surgeon’s control, and in some embodiments can also support semi-autonomous functions to assist or safeguard the procedure.

A feature of the system is a multi-degree-of-freedom tactile sensor array distributed along the cobot’s end-effectors (e.g., surgical grippers, dexterous hands or instrument tips). The tactile sensors detect forces, pressure, tissue compliance, texture, and other contact parameters in real time during the procedure. This rich tactile data (for example, sensing subtle slip of a grasped object or the contour and firmness of tissue) is continuously collected and relayed to the control system. The cobot’s control system processes these sensor signals to create a live feedback stream representative of the touch and force interactions occurring at the surgical site.

Complementing the tactile sensing is a haptic feedback generation system that reconstructs the sensed touch sensations for the remote surgeon. The surgeon’s control console is equipped with one or more haptic interface devices, such as force-feedback gloves, exoskeletal controllers, or haptic joysticks, that actively reproduce the forces and vibrations captured by the cobot’s sensors. In use, the surgeon manipulating the console’s input devices will feel sensations corresponding to the remote surgical environment, including resistances when the robot encounters tissue, vibrations or impacts from tool-on-tool contacts, and even the slight give of tissue deformation. This immersive haptic feedback loop allows the surgeon to experience a realistic sense of touch, greatly enhancing precision and situational awareness during telesurgery.

The surgical control console with immersive interface provides an integrated experience combining visuals, touch, and precise control. High-definition stereoscopic video or other sensor feeds from cameras on the cobot give the surgeon a clear view of the operative field. The console’s haptic devices convey touch feedback from the cobot as described, and the console’s master control devices (e.g., hand controllers or instrument replicas) translate the surgeon’s hand movements into scaled, precise movements of the robotic instruments. The interface may allow the surgeon to adjust sensitivity settings, such as scaling down or up the force feedback and motion ratio. For example, force scaling and motion dampening settings can be tuned so that large movements or forces by the surgeon are translated into finer, gentler actions by the robot, aiding delicate maneuvers. Tactile feedback filtering can also be adjusted to reduce noise or emphasize certain sensations, according to surgeon preference or the requirements of the procedure.

To connect the remote console with the patient-side robot, the system utilizes a latency-minimized communication architecture. A high-bandwidth, low-latency network link (wired or wireless, such as a dedicated 5G or fiber-optic connection) carries control signals from the console to the cobot and returns sensory and video data from the cobot to the console in real time. This bidirectional link ensures that the surgeon’s commands are executed immediately and that haptic/visual feedback is delivered without perceptible delay, which is critical for maintaining an intuitive teleoperative experience. The communication system may incorporate buffering and predictive algorithms to compensate for any brief network disruptions or latency spikes. In practice, if network latency momentarily increases or packets are lost, the system can momentarily interpolate or predict motions and feedback to prevent sudden jumps or loss of haptic fidelity, thereby preserving a smooth and continuous operation even under less-than-ideal network conditions.

The cobotic system further includes adaptive force control and safety protocols specifically designed for telesurgical use. Force-limiting algorithms in the control software monitor the magnitude of forces and torques detected at the robotic end-effectors and dynamically enforce safety thresholds. If excessive force is being applied (for example, if a gripper inadvertently clamps down too hard on tissue), the system automatically scales down or halts the motion to prevent damage to delicate anatomical structures. In addition, the control system monitors for any anomalous tactile patterns or resistance that does not match the expected input (such as a sudden obstruction or misalignment of a tool). Upon detecting an unexpected hard stop or unusual resistance, the system can immediately pause or constrain robotic movement, alerting the surgeon and preventing potential injury or equipment damage. These safety compliance measures act as an active safeguard layer during remote operations.

The tactile feedback is also context-aware. The system can adjust the sensitivity and frequency of feedback based on the current surgical context, such as the phase of the procedure, the type of instrument in use, or the nature of the tissue being interacted with. During high-precision tasks (for instance, a delicate nerve dissection or micro-suturing of a vessel), the system may automatically enhance the tactile resolution and reduce motion scaling, allowing the surgeon to feel finer forces and make more precise movements. Conversely, during gross tissue manipulation or when using larger instruments, the feedback might be modulated to prevent overwhelming the surgeon with unnecessary detail. This context-aware feedback modulation ensures that the surgeon receives the most relevant tactile information for each step of the procedure, improving both precision and comfort. Moreover, cameras on robotic end effectors can aid in the spatial positioning of the end effectors and maximize fidelity when coupled with haptic feedback.

Another aspect of the system is real-time procedural logging and data analysis. The cobot’s control system continuously records key operational data throughout the remote surgery. This includes the surgeon’s control inputs (movements, commands issued), the cobot’s responses, tactile sensor readings, haptic feedback signals, and associated video feeds of the surgery. All haptic interactions, for example, every instance of contact, force applied, or resistance encountered, are logged with timestamps. The system flags noteworthy events, such as moments where force thresholds were approached or exceeded, or when the safety algorithms intervened to halt a movement. These detailed logs can be used for postoperative analysis and training: surgeons can review their performance with synchronized video and force data, and surgical trainers or auditors can evaluate adherence to protocols or investigate any irregular events. Additionally, the recorded data supports regulatory documentation and quality assurance, providing an objective record of what occurred during the telesurgical procedure.

The perioperative cobotic system is also engineered for sterile-field compliance and general surgical safety despite the physical separation of the surgeon. The patient-side robotic platform is designed to operate in an operating room without violating sterile boundaries, for instance, the robotic arms can be draped or sterilized as needed, and the system’s movements are constrained so as not to collide with the patient, operating table, or personnel. It includes proximity sensors and software rules to maintain a safe distance from the surgical site except where intervention is intended. The system supports remote accessibility in that a surgeon can operate either from a control room within the same facility (e.g. a neighboring room outside the sterile OR) or from a geographically distant location via a secure network. The communication and control protocols are compatible with standard telepresence and hospital IT infrastructure, ensuring that the remote link is secure and reliable. Furthermore, multiple fail-safes are built in: for example, an emergency stop function can be triggered by the surgeon or automatically if a critical fault is detected, immediately freezing the robot’s motion. In the event of a network failure or console disconnect, the cobot can either enter a safe hold state or execute a pre-programmed safe retraction to a standby position to protect the patient.

Overall, the disclosed cobotic system provides a comprehensive solution for telesurgery by integrating advanced tactile sensing, haptic feedback, immersive remote operation, adaptive control, and safety mechanisms. By transmitting the sense of touch across distances, the system allows surgeons to carry out precise surgical interventions on a patient in one location while physically operating from another, with a level of control and sensory awareness approaching that of hands-on surgery. This innovation expands the capabilities of remote surgery, enhancing surgeon dexterity and confidence, while maintaining patient safety and broadening access to specialized surgical expertise regardless of location.

### BRIEF DESCRIPTION OF DRAWINGS

The foregoing and other objects, features, and advantages of the invention will become more apparent from the following detailed description, taken in conjunction with the accompanying drawings, in which:

FIG. 1 is a perspective view of a perioperative cobotic system configured for remote surgical interventions.

FIG. 2 is a functional block diagram illustrating an example system architecture of the perioperative cobotic system.

FIG. 3 is a diagram illustrating the distribution of tactile, force, and proximity sensors along a robotic arm and an associated closed-loop tactile feedback pathway.

FIG. 4 is a diagram illustrating a haptic feedback generation system and a surgeon control console configured to provide immersive tactile and visual feedback to a remote surgeon.

FIG. 5 is a diagram illustrating an example communication architecture for bi-directional transmission of control commands and sensory feedback with latency management and predictive buffering.

FIG. 6 is a schematic view of a surgeon control console including haptic control devices, visual display interfaces, and user-adjustable feedback controls.

FIG. 7 is a diagram illustrating force-limiting and compliance control logic used to monitor and regulate forces applied by the robotic arms during surgical procedures.

FIG. 8 is a flowchart illustrating an example method for context-aware modulation of tactile and haptic feedback based on surgical phase, tool type, or anatomical context.

FIG. 9 is a diagram illustrating real-time procedural logging and event tagging of tactile data, control inputs, and video during a remote surgical procedure.

FIG. 10 is a diagram illustrating integration of the perioperative cobotic system within a sterile surgical environment and remote accessibility of the system by a surgeon.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

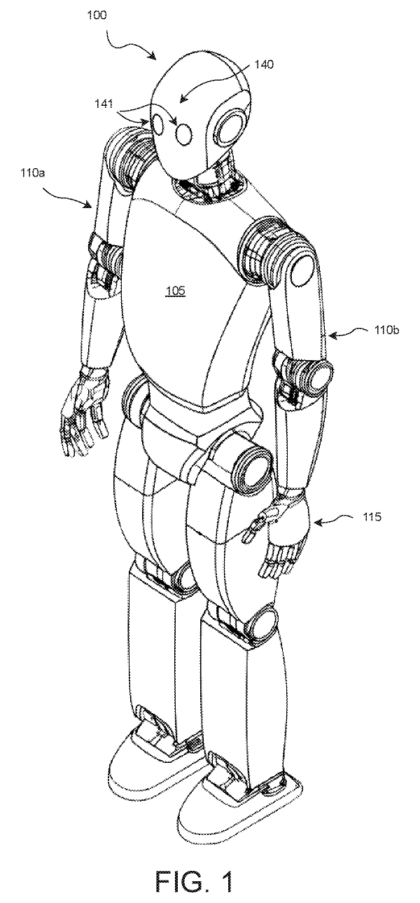

At Referring now to FIG. 1, a perioperative cobotic system 100 is shown in a perspective view with its major hardware components. In the illustrated embodiment, the system 100 includes a base platform supporting dual multi-jointed robotic arms 110a, 110b. The base may be a mobile cart or a fixed pedestal forming a stable torso-like structure. Each robotic arm 110a, 110b provides multiple degrees of freedom for surgical manipulation (e.g., joints analogous to a human shoulder, elbow, wrist, etc.), enabling precise instrument handling and dexterous tasks. At the distal end of each arm is an end-effector 115 (such as a gripper or tool interface) capable of holding surgical instruments and interacting with patient tissues. The end-effectors 115 are equipped with dexterous manipulators (articulated fingers or grippers) and incorporate integrated tactile sensors and cameras to detect touch, pressure, and texture of contacted objects. In this embodiment, the cobotic platform 100 has two arms to facilitate bimanual operation, allowing a remote surgeon to perform coordinated tasks (for example, one arm retracting tissue while the other operates a surgical tool) in a manner similar to an in-person surgeon using both hands.

The cobotic system 100 is designed to operate within an operating room’s sterile field. All exposed surfaces of the robot are smooth and sterilizable, and in some cases covered by a sterile drape or removable sterile sleeves. As shown in FIG. 1, a smooth outer shell 105 may encapsulate the base and arm structures to prevent contamination; joints and seams are sealed or easily draped so that the device can be introduced into a sterile surgical field without introducing bioburden. The outer materials and coatings are chosen to withstand common sterilization procedures (e.g., autoclave cycles, chemical disinfectants) or to be used with disposable sterile covers. This design consideration enables the cobot to work directly adjacent to the patient and surgical staff. FIG. 1 further illustrates that one or more sensor modules 140 are distributed on the cobot’s body, for example, a sensor cluster (such as cameras or depth sensors) may be mounted on a head or mast of the base to provide visual oversight of the surgical site. In the depicted embodiment, stereoscopic cameras 141 and microphones (not explicitly shown) can be positioned on an upper section of the platform to capture real-time video and audio of the operating field for the remote surgeon’s benefit. The sensor suite 140 and robotic arms together enable the system 100 to effectively stand in for a human presence at the operating table, performing physical interventions while relaying rich sensory information back to the surgeon.

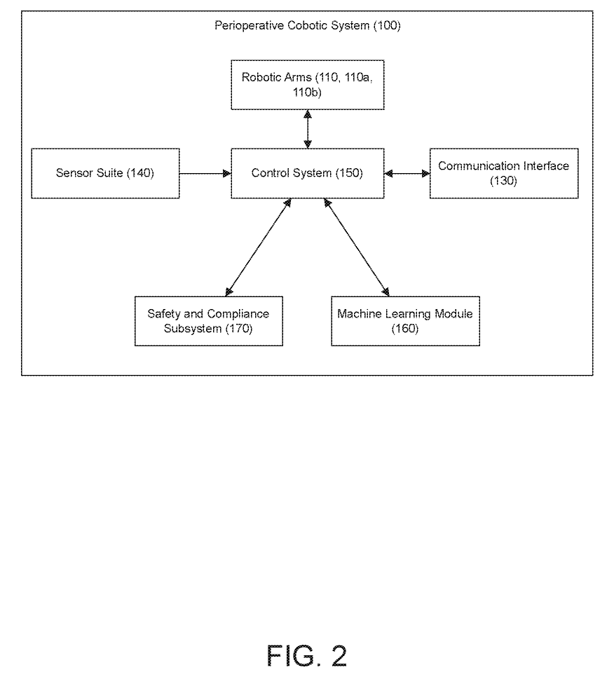

FIG. 2 is a schematic block diagram illustrating the architecture of the perioperative cobotic system 100 and its control interfaces. The central control system 150 is housed within the cobot (e.g., in the base or torso) and is operatively connected to all robotic actuators and sensors. The control system 150 may include one or more processors, motor controllers, and real-time control circuits responsible for coordinating movements of the arms 110a, 110b in response to surgeon commands. In the block diagram, each robotic arm 110 is driven by the control system 150 via low-level actuator controllers that govern joint motors, while receiving feedback from joint encoders and force sensors in a closed-loop manner. The sensor suite 140, including both tactile sensors and other modalities (imaging cameras, force/torque sensors, etc.), feeds data into the control system 150 for processing. For example, force readings from sensors at the arm’s joints and end-effectors are continuously sent to control system 150 to enable active force feedback control, and high-definition video from the cobot’s cameras is streamed into the control system for transmission to the surgeon’s console (as described further below).

As shown in FIG. 2, a communication interface 130 links the local cobot control system 150 to a remote surgeon’s station (console). The communication interface 130 encompasses the hardware and protocols for bidirectional data exchange between the operating room (where system 100 is located) and the remote operator. In one embodiment, interface 130 utilizes a low-latency, high-bandwidth network link, for instance, a dedicated high-speed internet connection or private 5G network, to carry two primary data streams. A first stream carries control commands from the remote surgeon to the cobot (e.g., movement commands for the arms, tool actuation signals, camera angle instructions), and a second stream carries feedback data from the cobot back to the surgeon (including live video feeds, audio, and haptic feedback signals derived from the cobot’s tactile sensor readings). To ensure reliability and minimal delay, the communication interface 130 can employ multiple redundant channels or communication technologies. For example, a real-time channel (such as a wired fiber optic line or ultra-fast wireless link) may be dedicated to time-sensitive data like haptic feedback and control commands, while a parallel high-throughput channel handles video streaming and less time-critical data. The interface 130 preferably maintains an encrypted link for data security, given that patient information and remote control signals must be protected within hospital IT infrastructure. In practice, the control system 150 and communication interface 130 cooperate to synchronize the remote surgeon’s inputs with the robot’s actions in near real-time, creating a seamless teleoperation experience. The system architecture of FIG. 2 thus includes functional modules for motion control, sensory processing, communication, and feedback, all tightly integrated to support remote surgical intervention.

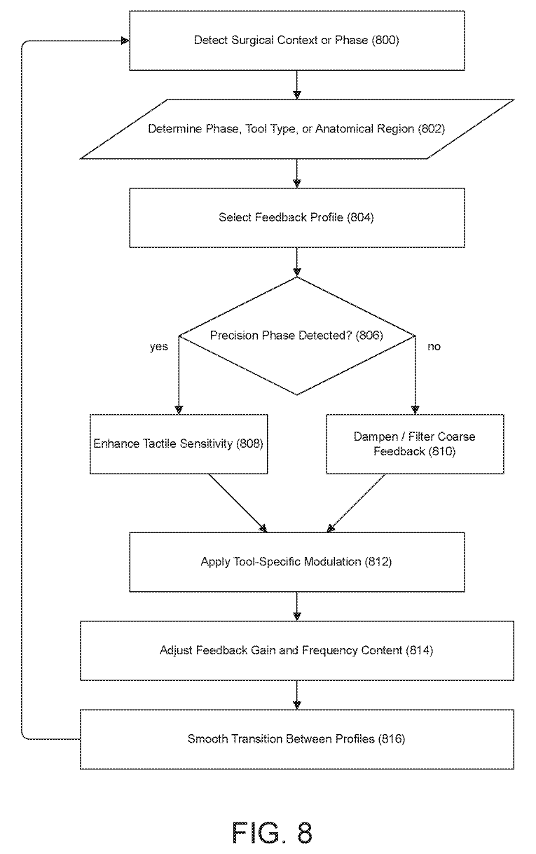

Within the control system 150, additional specialized subsystems contribute to intelligent and safe operation. A machine learning module 160 is interfaced with the control logic and sensor inputs, as indicated in FIG. 2. The machine learning (ML) module 160 processes data from the sensor suite 140 (and optionally from the surgeon’s command patterns) to provide higher-level context awareness and predictive capabilities. For instance, the ML module 160 can analyze the live video feed or instrument motion data to recognize the current surgical phase or detect anatomical structures in view. Based on learned patterns (trained on historical surgical data or simulations), module 160 may predict the surgeon’s next intended action or detect subtle changes in tissue interaction. These predictions enable the system to proactively adjust control parameters or prepare relevant feedback. As an example, if the ML module 160 infers that the procedure is transitioning from a coarse dissection phase to a delicate suturing phase (perhaps by recognizing a particular instrument being used or a change in motion finesse), it can signal the control system 150 to increase the sensitivity of force feedback and apply finer motion scaling (this context-aware adjustment is described further with respect to FIG. 8). The ML module 160 can be implemented as a suite of algorithms, including neural networks trained on surgical kinematics and tactile datasets, running either on the cobot’s onboard processors or on a connected computing unit. In some embodiments, part of the ML processing may occur at the remote console or a cloud service, with results sent back to the control system 150, so long as latency is kept within acceptable bounds.

Also depicted in FIG. 2 is a safety and compliance subsystem 170 that acts as a monitoring and override layer for the entire system. The safety subsystem 170 receives real-time data streams from sensor suite 140 (such as distances from proximity sensors, force levels from tactile sensors, joint torques, etc.) and supervises the commands issued by control system 150. If a potentially unsafe condition is detected, the safety subsystem 170 can intervene within milliseconds to modify or override robot motion. For example, if the cobot’s arm 110a is moving toward a region outside the permitted surgical area, or if an unexpected obstacle (like a human hand or an instrument) enters the cobot’s workspace, the safety logic will constrain the motion, slowing it or halting entirely, to prevent a collision. Similarly, if a force sensor on an end-effector registers a sudden spike indicating an unexpected resistance (perhaps the instrument pressed on a harder-than-anticipated structure or got snagged), the safety subsystem 170 can immediately freeze or retract the arm to avoid exerting excessive force on delicate tissues. In normal operation, the control system 150 alone dictates the actuation commands, but the safety subsystem 170 runs in parallel as a check: it continuously evaluates whether all parameters stay within predefined safe limits (e.g., joint speeds below a max, forces below thresholds, position within a safe zone, network latency below a threshold, etc.). Should any parameter deviate beyond safe bounds, safety subsystem 170 will either correct the action or trigger an emergency stop protocol, depending on the severity of the condition. The architecture in FIG. 2 is preferably designed with redundancies and fail-safes for safety, such as independent processing threads for safety monitoring and backup communication links, ensuring that a single fault cannot lead to loss of control or awareness. All modules (control 150, ML 160, comm 130, safety 170, etc.) interact continuously to enable a robust, real-time remote surgery system, as will be described in further detail in connection with subsequent figures.

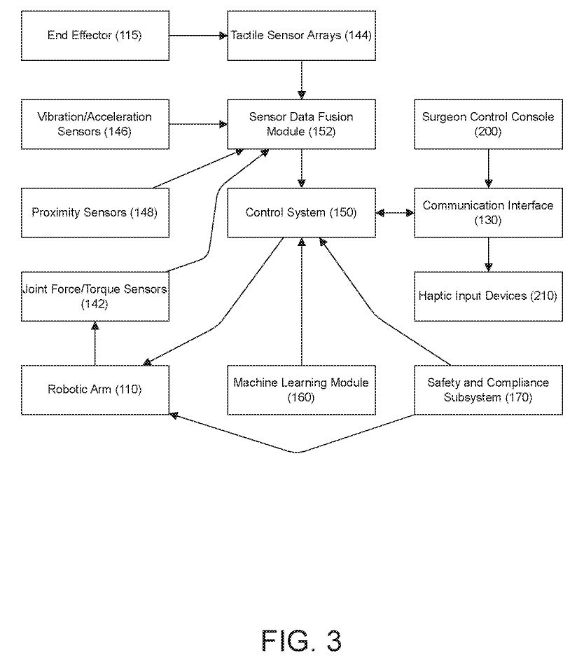

FIG. 3 illustrates the distribution of sensors along the robotic manipulators and how tactile data flows through the system in a closed loop. Each robotic arm 110 of the system carries a variety of sensors at strategic locations to capture forces, pressures, and other tactile information during surgical interaction. As depicted in FIG. 3, joint-level force/torque sensors 142 are installed at one or more joints of the arm (for example, at the wrist joint or between arm segments) to measure torques and loads experienced by the arm in three dimensions. These provide baseline feedback on forces exerted by the arm as it moves or when it encounters resistance. Additionally, tactile sensor arrays 144 are integrated near the end-effectors 115, for instance, on the inner surfaces of gripper fingers or on the tip of a tool adaptor, to detect pressure distribution, texture, and slip at the immediate contact interface with tissue or instruments. In one embodiment, each finger of the gripper has an array of pressure-sensitive pads or MEMS tactile sensors capable of sensing fine details like the difference between soft organ tissue and firmer material (such as a surgical needle) by measuring contact force patterns. Some implementations may also include vibration sensors 146 or accelerometers on the end-effector to pick up high-frequency vibrations (e.g., the feel of a tool vibrating when it contacts bone or when an ultrasonic instrument is activated). These various sensors, force/torque sensors 142, tactile arrays 144, vibration/acceleration sensors 146, together form the multi-degree-of-freedom tactile sensor array of the system (part of the overall sensor suite 140).

All sensor signals are routed through the arm and body to the control system 150 in real time. As indicated by the data flow arrows in FIG. 3, each sensor’s output is digitized and streamed into a sensor data fusion module 152 within the control system. This module time-synchronizes and aggregates the tactile data along with other sensor inputs (such as visual data from cameras, if relevant) to create a coherent picture of what the robotic arms are “feeling” and doing at any given moment. For example, during a tissue grasping action, the system simultaneously knows the precise force applied by the gripper (from sensors 142/144) and can detect incipient slip if the pressure at the fingertips changes rapidly, a condition that might prompt the system to adjust grip force slightly or alert the surgeon. The tactile data flow is designed to be high-bandwidth and low-latency: sensors 142, 144, 146 may report at rates of hundreds or thousands of samples per second, ensuring that even brief or subtle interactions (like a faint tremor or a small bump encountered by the instrument) are captured and relayed.

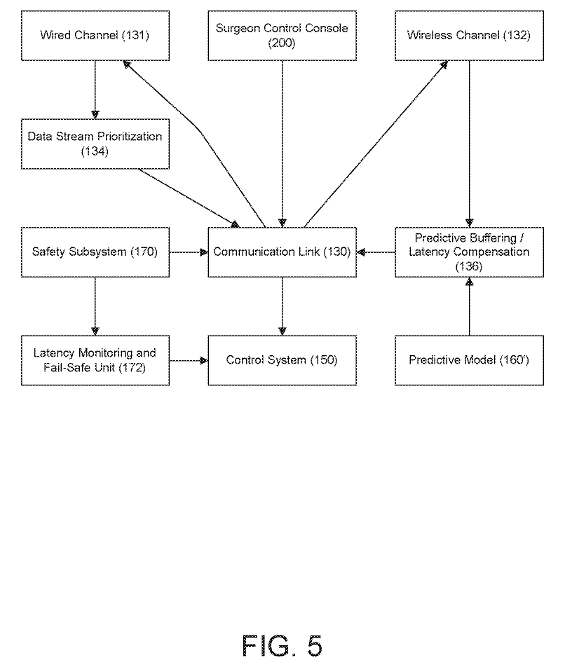

Within the control system 150, a portion of the tactile data is used locally for immediate control adjustments (e.g., local force control loops to modulate actuator output to maintain a desired force), while the full tactile dataset is packaged and sent through the communication interface 130 to the remote surgeon’s console as part of the haptic feedback stream. FIG. 3 emphasizes the closed-loop nature of this tactile feedback pathway: when the surgeon commands a movement (via the console, as will be described later), the robotic arm 110 executes that movement; any forces encountered are detected by the sensors (142, 144, etc.), fed into control system 150, and then almost instantly transmitted back to the surgeon’s haptic interface. The surgeon thus feels a force or resistance corresponding to what the robot arm experiences. This closed-loop feedback pathway is tuned for stability and responsiveness. In one embodiment, the haptic feedback loop (from sensor detection to force feedback at the surgeon side) is updated on the order of 1 kHz, which approaches the sensitivity of human touch and allows the remote operator to feel smooth, continuous forces rather than choppy or delayed feedback. To achieve this, the system minimizes any buffering in the tactile data flow and leverages the high-speed communication link 130 (as detailed in FIG. 5) to keep the feedback loop delay well under a few dozen milliseconds.

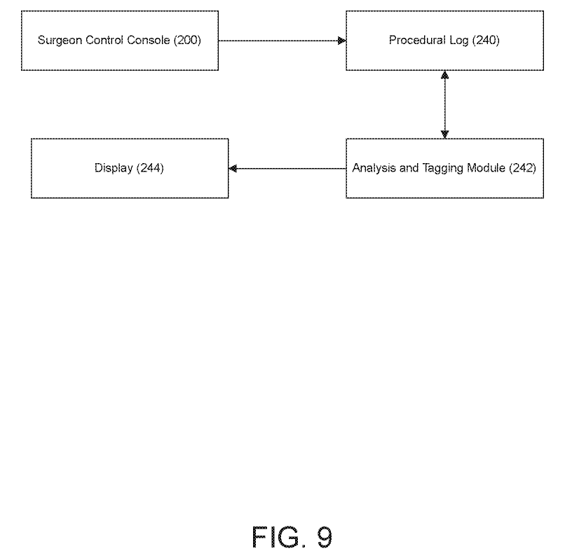

FIG. 3 also shows additional sensor types that may be present along the arms to support both haptic feedback and safety: for instance, proximity sensors 148 (such as infrared, optical or ultrasonic distance sensors) can be placed near the elbows or wrists of the arms to detect nearby obstructions or the patient’s body, providing another layer of data to avoid unwanted contact. These sensors work in tandem with tactile sensors: whereas a tactile pad 144 tells the system when and how it has touched something, a proximity sensor 148 can warn just before contact occurs. All such data flows into the control system 150 where dedicated algorithms decide how to respond, be it to initiate haptic feedback, adjust grip, stop motion, or simply log the event. The result is a rich stream of tactile and force data flowing from the robotic arms 110 and being utilized both for immediate control (locally and via the surgeon’s haptic device) and for higher-level analysis and logging (as discussed with respect to FIG. 9).

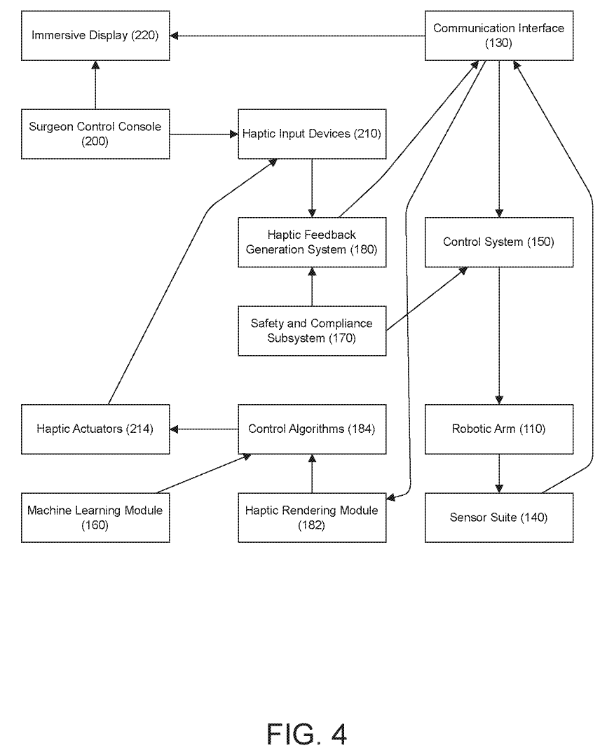

FIG. 4 illustrates the haptic feedback generation system and the surgeon’s interface for experiencing tactile sensations from the remote surgical site. The remote surgeon operates from a surgeon’s control console 200 which includes one or more haptic input devices 210 and an immersive display 220. The haptic input devices 210 are the primary controls that the surgeon manipulates to control the robotic arms 110, and they are configured to provide force and tactile feedback to the surgeon’s hands. In various embodiments, the haptic devices 210 may take the form of force-feedback joysticks, master controller arms, or even wearable haptic gloves or exoskeletal hand controllers. For example, in one implementation, each of the robot’s arms 110a, 110b corresponds to a scaled-down master arm or articulated controller 210a, 210b that the surgeon holds onto; movements of the master controllers are sensed and translated into corresponding movements of the robot arms (with appropriate scaling and filtering), while motors in the controllers push back on the surgeon’s hands to mirror the forces that the robot encounters. In another implementation, the haptic interface 210 could be a pair of instrument-shaped controllers that replicate the feel of actual surgical tools, when the surgeon “squeezes” a virtual forceps on the console, the real robotic forceps grasps tissue, and any resistance is fed back as force against the surgeon’s fingers on the controller.

The haptic feedback generation system 180 (represented schematically in FIG. 4) is the component that converts raw sensor data from the cobot into realistic tactile sensations for the remote operator. This system 180 comprises software algorithms and actuation hardware. On the software side, a haptic rendering module 182 within the control system (or console computer) interprets the force/pressure data coming from the sensor suite 140 and computes the corresponding feedback forces to apply to the surgeon’s device 210. This involves scaling and filtering logic to ensure stability and realism: for example, if the cobot’s gripper detects a certain force, the system can apply a proportional force on the surgeon’s controller, potentially scaled down or up based on user preferences or safety limits. The rendering module 182 may simulate different tactile effects, such as vibration, resistance, or impacts, by modulating the feedback signal. For instance, a rapid vibration in the robot’s tool (perhaps due to an ultrasonic instrument) can be reproduced as a buzzing sensation in the surgeon’s hand controllers, and a sudden bump or collision the robot experiences might be rendered as a jolt or impulse force on the controls. On the hardware side, the haptic interface devices 210 themselves contain actuators 214 (electric motors, force-feedback actuators, or pneumatic cylinders, depending on the design) that physically output forces to the user’s hands.

FIG. 4 illustrates that each degree of freedom of the input device (e.g., the pivot of a joystick, the open/close of a gripper handle, the roll of a wrist control) is paired with a controllable actuator that can dynamically create resistance or movement.

The haptic feedback system operates in closed loop with the tactile sensors, as mentioned earlier. FIG. 4 shows a flow chart of this real-time loop: (1) the surgeon’s input motion (through device 210) is sent as a command to the remote robotic arm 110; (2) the robotic arm interacts with the surgical environment (e.g., instrument contacts tissue); (3) sensors on the arm detect the interaction forces; (4) the sensor signals are transmitted back over the network; and (5) the haptic actuators 214 at the console apply the corresponding feedback to the surgeon’s hands. This loop repeats continuously at a high update rate. The system is configured to maintain stable haptic behavior even in the presence of network delays or any mismatch, for example, advanced control algorithms 184 (such as impedance control or admittance control algorithms) are implemented to ensure that the surgeon’s device does not oscillate or become unstable when encountering stiff contacts through the robot. These algorithms adjust the feedback in real time to account for the dynamic properties of both the robot and the human operator’s hands.

FIG. 4 also highlights features for adjustability and calibration of the haptic feedback. The surgeon can adjust settings on the console 200 to personalize the feel: for instance, force scaling can be tuned (the system might allow the surgeon to feel forces at a 1:1 ratio with the robot, or scaled down so that extremely high forces are dampened for comfort and safety), and motion scaling/dampening can be configured (e.g., the surgeon’s large hand movement might translate to a smaller precise movement of the robot, with some damping to smooth tremor). A tactile filtering option can filter out high-frequency noise in the feedback, for example, if there is a lot of vibration but the surgeon prefers to only feel gross forces, the system can be set to deliver a “filtered” haptic signal. These adjustments are part of the immersive interface and can be changed on the fly or preset for different procedure types. The haptic generation system 180 takes all these factors into account when computing the feedback forces. In summary, FIG. 4 shows how the system reconstructs a realistic sense of touch for the remote surgeon: by mapping tactile sensor data to haptic actuators in a tightly coupled loop, the surgeon can effectively “feel” the remote operative site as if physically present, gaining intuition about tissue consistency, tool engagement, and other tactile cues that are essential for delicate surgical maneuvers.

FIG. 5 depicts the communication architecture employed to minimize latency and ensure synchronized operation between the remote surgeon’s station and the local cobotic system. The diagram in FIG. 5 shows the flow of data through a network communication link 130 and various latency-reduction strategies built into the system. A primary design goal of the communication architecture is to maintain an end-to-end latency below a predetermined threshold (for example, on the order of only a few tens of milliseconds) so that the surgeon’s real-time control and haptic perception remain nearly instantaneous and natural.

As illustrated, the communication link 130 consists of both hardware components and protocol layers optimized for speed. On the hardware side, the system can leverage a combination of fiber-optic wired connections (providing very low transmission delay and high bandwidth) and wireless links (providing mobility and flexibility). In one embodiment, the operating room is equipped with a high-speed wired connection to a local network hub, and the remote surgeon’s console 200 is connected via a dedicated fiber line or a high-speed internet backbone to that hub. If the surgeon is within the hospital campus, a direct fiber or gigabit Ethernet link may be used. If the surgeon is remote (e.g., in another city), the system may use a reserved broadband connection or next-generation cellular network (5G/6G) with guaranteed bandwidth. FIG. 5 shows both a wired channel 131 and a wireless channel 132 as part of the communication interface; the system can dynamically choose the channel or use them in parallel for redundancy, for instance, sending critical haptic data redundantly over both paths and using whichever arrives first. This multi-channel communication approach helps avoid single points of failure and mitigates variable conditions (such as wireless interference).

On the software and protocol side, the communication architecture uses several techniques to minimize effective latency. One such technique indicated in FIG. 5 is data stream prioritization 134. Control signals and haptic feedback packets are given the highest priority, meaning they are sent and routed with minimal buffering. The video feed, which consumes higher bandwidth but can tolerate a slight buffering without loss of surgeon control, may be slightly compressed or have a one- or two-frame buffer to ensure it doesn’t congest the network. In practical terms, a separate real-time protocol (RTP or a custom UDP-based protocol) might carry the critical control/haptic data, while a secondary channel (possibly TCP or a streaming protocol) carries the video and audio. The system ensures these streams remain synchronized by time-stamping data and using synchronization algorithms, but will sacrifice a frame of video if needed to keep the control loop tight.

Another aspect shown in FIG. 5 is the use of predictive buffering and latency compensation 136. Because even a very fast network may have occasional jitter or packets arriving out of order, the control system 150 and ML module 160 incorporate prediction algorithms that can estimate the next likely control input or feedback signal in the brief event of a communication delay. For example, if the surgeon is moving an instrument in a steady direction and a single control packet is delayed, the system can predict that motion will continue for that fraction of a second and keep the robot moving smoothly, rather than pausing. Similarly, on the feedback side, if a haptic feedback packet is delayed, the console can temporarily extrapolate the force based on the last known sensor trend (e.g., if resistance was increasing, assume it continues to increase slightly) until the updated data arrives a few milliseconds later. This is represented as a predictive model 160′ in FIG. 5 working alongside the communication stack. Notably, the machine learning module 160 in the system may be trained to perform this predictive smoothing: it can analyze patterns of surgeon input and typical network behavior to make more sophisticated predictions beyond linear extrapolation (for instance, anticipating a deliberate stop or reversal of motion if that fits the surgeon’s usual technique in a given scenario). By employing these predictive buffering techniques, the system preserves a continuous feel of control even under transient network issues, effectively masking minor latency spikes from the surgeon’s perception.

FIG. 5 also shows a latency monitoring and fail-safe unit 172 as part of the safety subsystem. This unit continually monitors round-trip communication latency and data integrity. If a significant network latency spike or dropout occurs (e.g., if the latency exceeds a safe threshold Y for more than a brief moment, or if packets stop arriving altogether), the safety subsystem can automatically engage a controlled response. In one embodiment, upon detecting a latency anomaly, the safety controller 170 will momentarily halt or slow the robot’s motion (entering a “safe” mode where the arms maintain their last commanded position or move very slowly) and alert the surgeon to the connection issue. The system can also switch to an autonomous hold mode: for example, maintaining gentle holding of tissue or continuing a preprogrammed motion at a very slow speed until command is restored, rather than abruptly stopping in a way that could be unsafe. Once the link quality returns to normal, teleoperation control is smoothly re-engaged. These measures ensure that a network glitch does not lead to an uncontrolled situation. In summary, FIG. 5 illustrates how the system’s communication architecture is engineered for ultra-low latency and high reliability through fast channels, prioritized data, predictive compensation, and active monitoring, thereby preserving synchronous operation between the remote surgeon’s inputs and the on-site robotic actions at all times.

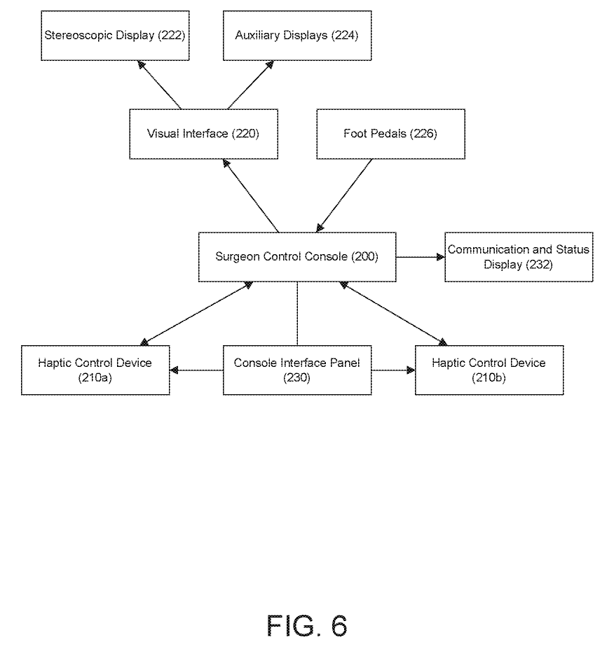

FIG. 6 provides a diagramatic view of the remote surgical control console 200, which features immersive visual and haptic interfaces for the surgeon operator. The console 200 is depicted as a comprehensive workstation that may include a combination of displays, input devices, and ergonomic controls enabling the surgeon to perform remote interventions as naturally as if present in the operating room.

Central to the console is the visual interface 220, which may consist of high-resolution monitors, stereoscopic displays, or even a head-mounted display or virtual reality (VR) headset. In the embodiment of FIG. 6, the console has a large stereoscopic monitor 222 that shows a live video feed of the surgical field in 3D (using input from the cobot’s cameras 141). This gives the surgeon depth perception and a clear, real-time view of the operative site. Optionally, the visual interface can include an augmented reality overlay on the video feed (for instance, indicating instrument trajectories or highlighting anatomy), though in the present system the emphasis is on haptic feedback; AR elements, if used, are integrated in a way to complement tactile cues (for example, an overlay might mark a region where high force should be avoided, providing a visual cue alongside the tactile sensation). The console may also include auxiliary displays 224 for vital signs, system status, or different camera angles, ensuring the surgeon has full situational awareness.

In front of the surgeon are the haptic control devices 210a, 210b (as described with FIG. 4). These hand controllers ergonomically positioned so the surgeon can comfortably manipulate them while watching the display. The console may include an adjustable chair and armrests to reduce fatigue, and the haptic devices 210 are mounted on a stable base that can be positioned to the surgeon’s preference. Each controller provides multiple input modalities: for example, foot pedals 226 or additional thumb joysticks could be present for secondary functions like camera control, tool selection, or issuing system commands (e.g., toggling between instruments or activating a cautery tool). All these input mechanisms are part of the immersive interface, allowing the surgeon to control various aspects of the operation without needing an assistant.

The haptic feedback delivered through devices 210 is calibrated at the console for the surgeon. FIG. 6 depicts a console interface panel 230 that allows the surgeon to fine-tune settings such as force feedback intensity, sensitivity, and any desired scaling. For instance, via the panel 230 or a touchscreen menu, the surgeon can select a “delicate mode” that scales down the forces (making the controls feel lighter and more sensitive, useful for microsurgery), or a “stabilization mode” that introduces slight damping to hand motions to filter tremors when doing extremely fine tasks. The console software may also let the surgeon replay or record short segments of the operation for immediate review (for example, to double-check an action via instant replay if something felt abnormal, tying into the logging discussed for FIG. 9).

Another element shown in FIG. 6 is the communication and status display 232, which provides feedback on system status, network health, and safety indicators. For example, a small indicator light or on-screen icon might show the network latency in real time (green for good, yellow for marginal, red for high latency), as well as any safety constraints currently active (if the robot’s safety subsystem has, say, limited arm speed due to proximity sensing, the console would alert the surgeon to that condition). The console thereby keeps the surgeon informed not just of the surgical view but also of the system’s own state.

Importantly, the remote console 200 is designed to be immersive, meaning the surgeon feels fully engaged in the remote environment. High-fidelity audio speakers or headphones may relay the sounds from the OR (e.g. equipment beeps or team communications), and a microphone allows the surgeon to speak to the OR team as needed. In some embodiments, the console 200 is enclosed or partitioned such that the surgeon has a distraction-free environment to focus on the surgery, with adjustable lighting and possibly even haptic transducers in the seat (for example, to feel large vibrations or to provide an alert via vibration if needed). All these features, illustrated in FIG. 6, contribute to the surgeon effectively feeling “telepresent” at the patient’s side. The combination of real-time 3D visuals, responsive haptic touch feedback, and intuitive controls allows the surgeon to carry out complex surgical interventions remotely with precision and confidence.

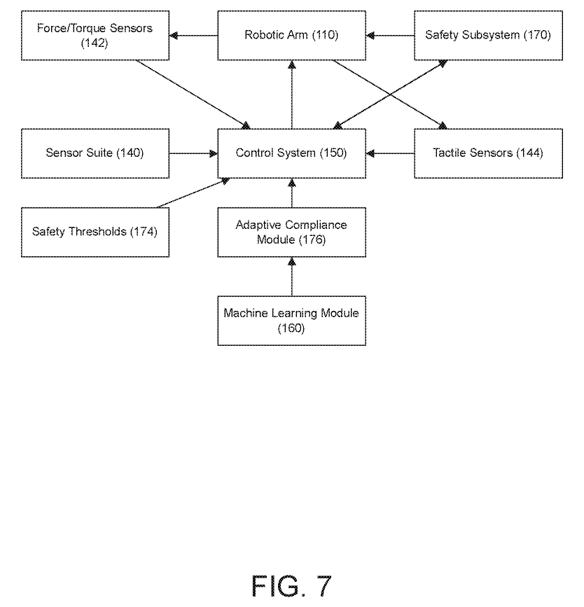

FIG. 7 illustrates the operation of the system’s force limiting and compliance control algorithms, which are critical for patient safety and delicate tissue handling. These algorithms run mainly within the control system 150 (with oversight by safety subsystem 170) and dynamically adjust the cobot’s behavior to prevent the application of excessive force and to ensure the robot responds compliantly when needed.

In the schematic of FIG. 7, the flow begins with the real-time force sensing from the sensor suite 140 (e.g., joint torque sensors 142 and tactile sensors 144 as described earlier). The measured forces and torques are continuously compared against predefined safety thresholds 174 stored in the system. These thresholds may be set based on empirical data about safe force limits for various tissues or instruments, for instance, a threshold for maximum force when handling a needle versus a lower threshold when manipulating a delicate organ. The force limiting algorithm uses these thresholds in a couple of ways. First, as a preventive measure, the control system 150 modulates actuator commands to keep forces within safe ranges. For example, if the surgeon pushes the controllers in a way that would command a high force (perhaps trying to cut tougher tissue), the system can automatically scale down the force output so that the actual force applied by the robot does not exceed a safe value (this is sometimes referred to as “force scaling” or virtual fixture limits in teleoperation). The surgeon might feel a stiff resistance in their haptic device once the limit is reached, effectively signaling that pushing harder will not translate into more force on the patient.

Second, the algorithm acts as a reactive safeguard: if an anomalous tactile pattern is detected, for example, a sudden spike or a force in an unexpected direction, the system interprets this as a potential problem (like hitting an unanticipated obstacle or the instrument getting caught). In response, as FIG. 7 shows, the safety subsystem 170 can instantly halt the current motion or even slightly retract the instrument. This happens on a fast control cycle (on the order of a few milliseconds) to minimize any risk of harm. A practical example: if the robot is inserting a needle and encounters a resistance that rises faster than a normal tissue puncture would, the system might freeze movement assuming something is wrong (perhaps the needle is bending or pressing on bone) until the surgeon assesses and gives a new input.

Compliance control is another facet illustrated in FIG. 7. “Compliance” in this context refers to the robot’s controlled flexibility or softness in response to forces. The system can adjust its control mode to behave in a more compliant manner depending on context. For instance, when the cobot is engaged in tasks like exploratory palpation of tissue or following along a surgeon’s guiding hand (in some training or cooperative mode), a high compliance setting is used: the arms will “give” easily under externally applied forces. Conversely, when holding a structure steady, compliance might be reduced to hold a firm position. The figure shows a block for adaptive compliance module 176, which takes input from the context (possibly from the ML module 160 or the detected surgical phase) to set the appropriate compliance level. During a sensitive phase like micro-suturing a fine vessel, the system might increase compliance in certain directions to avoid exerting lateral forces on the needle, so if any unintended force is felt, the arm moves slightly with it rather than pushing back. Alternatively, in a phase like moving or lifting an organ lobe, the system might enforce a lower compliance (stiffer control) but with strict force caps, so it can support the tissue without dropping it, yet not squeeze too hard.

Additionally, FIG. 7 includes a step where the system monitors force trends over time. This predictive aspect means the control system doesn’t just react to instantaneous force values, but also watches how quickly forces are building. If a force is ramping up quickly (even if below the limit at the moment), the control algorithm can preemptively slow the motion to prevent overshoot beyond the safe limit. This is akin to an electronic “shock absorber”: fast increases trigger dampening. The ML module 160 may assist here by distinguishing between expected force increases (e.g., the normal resistance felt when tying a suture knot) versus unexpected ones (like instrument entanglement).

All these mechanisms ensure that the cobot behaves in a safe and surgeon-friendly manner. From the surgeon’s perspective, these algorithms manifest as the sensation that the robot has a natural “give” if resistance is met, much like how a human surgeon’s hands would stop if something feels wrong. Meanwhile, in routine operation within normal force ranges, the algorithms are non-intrusive and transparent. Only when thresholds are approached do they intervene, often in subtle ways (scaling or damping) that do not disrupt the procedure. FIG. 7 thus encapsulates how the system enforces dynamic safety thresholds and compliance: by constantly comparing forces to safe limits, adjusting actuator outputs in real time, and responding instantly to any irregular force event to prevent harm to the patient or equipment.

FIG. 8 illustrates how the system modulates tactile and haptic feedback based on the surgical context or phase, providing context-aware adjustments to optimize the information conveyed to the surgeon at different times. The flowchart in FIG. 8 delineates several surgical phases and how the feedback parameters are tuned for each.

At the top level, the system either detects or is informed of the current surgical phase or task. This can be achieved automatically by the machine learning module 160 (using data such as instrument type in use, motion patterns, or visual cues to classify the phase of surgery) or manually by the surgeon/OR staff inputting a phase change (for instance, pressing a button when moving from incision to suturing). The phases might include, for example: incision, tissue dissection, suturing/closing, cauterization, etc., as well as idle or setup times.

For each phase, FIG. 8 shows adjustments along two main dimensions of feedback: tactile sensitivity and feedback frequency/content. During high-precision phases, e.g., fine suturing, nerve dissection, or microvascular work, the system increases the tactile resolution and sensitivity of the feedback. Practically, this means smaller forces or subtle textures are amplified so the surgeon can feel them distinctly. The system might lower the threshold for registering a feedback force, remove any minor filtering of signals, and ensure even faint tactile events (like a slight resistance as a needle passes through tissue layers) are transmitted. The surgeon’s haptic devices 210 might thus feel “lighter” and more responsive in the hand during these phases, enabling detection of very delicate cues. The figure denotes this as “enhanced feedback in precision phase.”

Conversely, in phases that involve gross manipulation or potentially a lot of background vibration (for example, sawing bone, using power tools, or repositioning large tissue sections), the system can moderate the feedback to prevent overwhelming the surgeon with too much sensation. In such phases, the control software might apply additional filtering to remove high-frequency jitter or dampen repetitive vibration that doesn’t convey useful information. The figure indicates “damping high-frequency feedback in coarse phase.” The rationale is that during a coarse task, the surgeon is likely already aware of the significant forces (which will still be fed back), but might not need to feel every fine vibration from, say, a power tool, which could be distracting or fatiguing. The system might also reduce the overall gain of the haptic feedback in these scenarios to avoid the surgeon feeling excessively strong forces continuously (since prolonged strong feedback can lead to operator fatigue or even loss of control precision).

Another context-aware adjustment shown in FIG. 8 relates to tool-specific feedback modulation. The system knows what instrument is being used (either by direct identification of the tool on the robot or by which controller/tool the surgeon selected). It can tailor the feedback accordingly. For instance, if a delicate micro-forceps is attached, the system assumes any force feedback should be subtle and precise, whereas if a large grasper or an orthopedic drill is attached, some heavier forces and vibrations will be expected. The control parameters (like feedback gain and frequency filtering) are thus loaded from a profile associated with each tool type. FIG. 8 represents this with a decision branch: if Tool A (delicate) is in use, then use profile A (high sensitivity, low dampening); if Tool B (heavy-duty) is in use, then use profile B (with more dampening, etc.).

Additionally, context can be derived from the target anatomy or tissue type being engaged. If the system (via imaging or prior knowledge from the surgical plan) recognizes that the robot is currently interacting with, say, a blood vessel or nerve (very delicate structures), it can sharpen the feedback and possibly impose more stringent force limits proactively (as a form of context-aware safety). If instead the robot is working with connective tissue or fascia (less delicate), normal feedback resumes. The ML module 160 might provide these insights by combining visual recognition and force signature analysis to guess the tissue type being manipulated.

All these adjustments happen seamlessly as the surgery progresses. FIG. 8 shows a loop where the system continuously monitors for phase changes or context shifts. When a shift is detected, the system fades the feedback settings from the old profile to the new one smoothly so as not to jolt the surgeon with a sudden change. For example, moving from dissection to suturing, the system might over a second increase the feedback gain and high-pass more of the fine forces through.

In summary, FIG. 8 depicts that the cobotic system is not static in how it presents haptic feedback; rather, it is intelligently context-aware. By modulating tactile feedback characteristics (sensitivity, filtering, frequency content) according to the current surgical activity, the system ensures the surgeon receives the most relevant and useful haptic information at each moment without unnecessary noise. This enhances precision when needed and reduces distraction or overload when it’s not, thereby improving the overall teleoperative experience.

FIG. 9 illustrates a procedural logging interface integrated into the system, and how haptic anomalies (or other notable events) are automatically or manually tagged during surgery for analysis and training purposes. In the diagram of FIG. 9, the flow of data from the operation is shown being captured and recorded in a structured manner.

The system continuously records a wealth of data throughout the telesurgical procedure. This includes: video streams from the surgical cameras, audio of communications, all the control inputs from the surgeon (movements of the input devices, button presses, etc.), and all sensor outputs (tactile sensor readings, forces, torques, positions of each joint over time). This comprehensive dataset forms the procedural log 240. The log is time-synchronized, meaning that for any given timestamp one can correlate what the surgeon was doing, what the robot was doing, and what it was sensing. In FIG. 9, this is represented by a timeline with multiple parallel tracks (video, control, haptic data, etc.), all aligned.

Key to FIG. 9 is the notion of event tagging, particularly for anomalies or threshold-crossing events. The system’s software includes an analysis and tagging module 242 that monitors the incoming data in real time. When certain conditions occur, it creates a tagged entry in the log. For example, if the force limiting algorithm (from FIG. 7) triggered an emergency stop or a significant force threshold was almost reached, the system flags that moment as a “force anomaly.” Similarly, if a sudden loss of contact was detected (e.g., something slipped from the gripper unexpectedly) or if the network latency fail-safe activated (as per FIG. 5), those moments get tagged. Each tag might include a brief description, timestamp, and relevant sensor readings. In FIG. 9, these appear as highlighted markers along the timeline (for instance, markers indicating “Force spike at 10:32:15” or “Latency dip at 10:35:47”).

Beyond automatic tagging, the interface also supports manual tagging by the surgeon or support staff. The console 200 may have a control (such as a foot pedal or a button on the UI) that the surgeon can press when they subjectively feel something noteworthy through the haptic feedback, in other words, if the surgeon senses an unusual texture or resistance (perhaps suspecting a tumor or a calcification in tissue, or just noting a haptic cue that was interesting), they can mark that point. The system will insert a tag like “Surgeon Flagged Event” at that timestamp. This is extremely useful for later review because it draws attention to moments that the human operator deemed important, even if they didn’t trigger an automatic threshold.

The interface in FIG. 9’s (which might be viewed on a computer after the procedure, or by an assistant in real time) allows users to click on or select these tagged events to quickly review what happened. For example, selecting a force anomaly tag could replay a short segment of the video and display a graph of force vs. time around that event, showing exactly how high the force went and how the system responded. The display 244 in FIG. 9 conceptually shows such a graph or readout, perhaps the force profile with a red line indicating the safety limit, and a peak that hit that line prompting a cutoff. For a surgeon-flagged tag, the system could present whatever note the surgeon attached (the surgeon might categorize it via a quick menu, e.g., “unusual tissue stiffness here”).

All recorded data including these annotations can be stored in a secure database or on the console for various purposes. One purpose is postoperative analysis: The surgical team can review the logs to understand any difficulties or near-misses. For instance, if there were multiple force spikes at certain steps, the team might discuss adjusting technique or tool selection for future cases. Another purpose is training and skill improvement: a surgeon in training could replay their teleoperative session, using the tags as pointers to where they might have, say, applied too much force or taken extra time. The haptic data could even be used to provide objective metrics (e.g., “maximum force applied”, “smoothness of motion” etc.).

Additionally, from a regulatory and documentation standpoint, this procedural log serves as a detailed record of the remote surgery, which might be useful for compliance with surgical protocols or in case of any adverse event reviews. The presence of haptic data adds a layer of detail not available in conventional surgery records, it’s like having a recording of the surgeon’s “sense of touch” throughout the case. This could contribute to evidence of due care or be analyzed to improve system algorithms.

Finally, these logs and tagged anomalies feed back into the system improvement loop. The machine learning module 160 can use historical logged data (with tags indicating, for example, where it predicted correctly or where an unexpected event occurred) to retrain models and improve future performance. For instance, if many cases log a certain type of force spike at a particular maneuver, engineers might adjust the force threshold or the ML might learn to anticipate that scenario earlier. FIG. 9 thus represents not just a logging tool, but a continuous improvement and feedback mechanism for the technology and its users, leveraging detailed tactile and control data from each procedure.

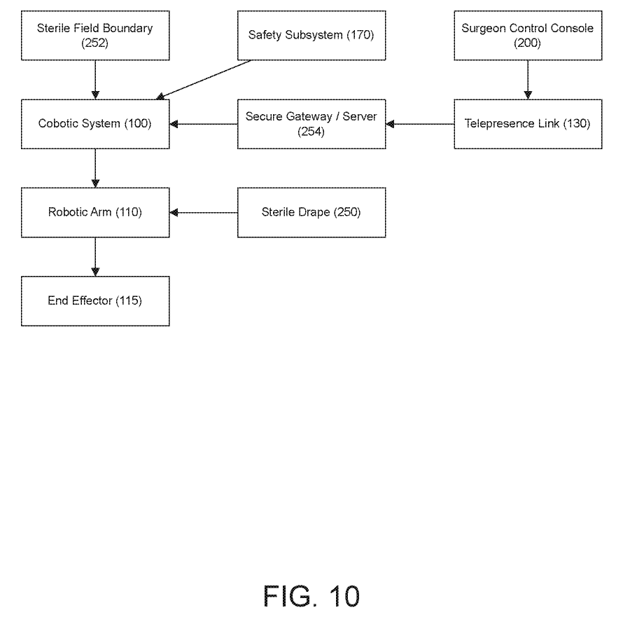

FIG. 10 illustrates how the cobotic system integrates into the sterile field of an operating room and how it enables remote accessibility for surgeons, including an overlay of remote operational elements onto the local surgical environment. In this conceptual diagram, the operating room setup is shown with the cobot system 100 positioned near the patient on the operating table, and indicators of the sterile field boundaries and remote connectivity.

To maintain sterile-field integration, the cobot is either draped with sterilized covers or constructed from sterilizable components, as discussed in FIG. 1. FIG. 10 highlights that the cobot can be placed such that its working envelope overlaps the surgical site without contaminating it. For example, if the cobot is mobile, it may dock at a fixed position where part of its structure (like the arm 110 and end-effector 115) extends into the sterile field over the patient, while its base remains outside or at the periphery. A sterile drape 250 can be shown in FIG. 10 covering the portions of the robotic arms that are above the patient. The system can include specialized draping kits that allow the arms to move freely while covered. In some embodiments, the end-effectors themselves are treated as surgical instruments: they are sterilized before attachment, and the robot can accept them in a manner that maintains sterility (for instance, an instrument exchange might be done by a scrub nurse attaching a sterile tool onto the robotic wrist under normal sterile technique).

The sterile field boundary 252 is situated around the patient and instrument table. The cobot’s safety system 170 is cognizant of this boundary, using either predefined coordinates, sensors (visual recognition of drapes or markers), or input from staff, and it will enforce that non-sterile parts of the robot do not cross into sterile zones. This may include software “no-go zones” beyond which the arms will not travel unless certain conditions are met. If the robot is overhead-mounted (one of the variant possibilities), it might actually reside entirely within the sterile field, essentially becoming a sterile fixture for the duration of surgery after being properly sanitized.

On the remote accessibility side, FIG. 10 shows that the system is connected through hospital networks (or broader networks) to enable surgeons in distant locations to operate. An icon or overlay might represent the remote surgeon’s presence, for instance, a display in the OR might show a video feed of the remote surgeon or an indicator of who is controlling the robot at the moment. The term “remote accessibility overlay” in FIG. 10 can be interpreted as an augmented element in either the OR or the remote UI that links the two sites. For example, in the OR, there could be a screen that highlights when the robot is under active remote control (perhaps with the remote surgeon’s name or avatar and telemetry info displayed, reassuring the local team of the connection status). Conversely, on the remote surgeon’s interface (FIG. 6), an overlay might show a floor plan or a schematic of the OR indicating the cobot’s location relative to the patient and staff, which helps the remote surgeon maintain orientation and awareness of the OR context.

The system supports various telepresence protocols: it can integrate with standard hospital IT systems for video conferencing, audio links, and data sharing. As illustrated in FIG. 10, a telepresence link 130 (conceptually the same as link 130, drawn to emphasize remote connectivity) not only transmits surgical control data but can also carry additional streams such as a live audio dialogue between the remote surgeon and the OR team, or additional camera views (like an overhead OR camera feed) so the remote surgeon knows, for instance, if a nurse is repositioning something or if there’s any non-verbal cue in the room.

Remote accessibility also means multiple remote participants can be given access. FIG. 10 may show, for instance, two remote consoles connected: one primary surgeon and another could be an assisting surgeon or an observer (perhaps a specialist giving consultation). The architecture can handle such multi-node connectivity securely. Each additional participant might have a tailored view, e.g., an observer could see the video and data but not control, whereas an assisting surgeon might take control of the second arm 110b from another console if needed. The system manages these roles and ensures that only authorized inputs affect the robot (with perhaps a “master” control designation that can be passed or shared in a controlled manner).

In terms of compliance with surgical protocols, the remote nature of the operation requires strict adherence to medical data security and patient privacy rules. The system, as part of hospital integration, ensures that all remote connections are encrypted and authenticated. FIG. 10 may symbolically represent secure gateways or servers 254 that handle this integration, effectively the cobot ties into the hospital network such that it appears as just another surgical device, albeit one controlled from afar. The system logs remote access sessions (who connected, from where, when), aligning with hospital requirements for audit trails especially in telemedicine scenarios.

An additional overlay shown in FIG. 10 could be data displayed to the local surgical staff about the robot’s status. For example, a small monitor on the robot or in the OR could show a simplified view of what the robot is “feeling” (like force readouts or status messages). This helps the co-located team trust and verify the robot’s actions. If the robot is about to move (under remote command), it might announce or display “Robot moving to position X” as a prompt so that people can step back if needed, effectively projecting the remote intent into the local space.

In summary, FIG. 10 demonstrates that the perioperative cobotic system is designed to seamlessly blend into the surgical environment both physically and procedurally. It maintains sterility and safety in the OR while extending the surgical reach to remote operators. The “overlay” aspect underscores that information flows both ways: the remote surgeon is virtually present in the OR through data and video, and the OR staff are aware of and integrated with the remote surgeon’s actions through visual/auditory indicators. This bidirectional integration ensures that even though surgery is being conducted across distance, it remains a coordinated and compliant surgical procedure under standard hospital protocols.

While the foregoing description and figures(FIGS. 1–10) illustrate a particular embodiment of a dual-arm humanoid-form cobotic surgery system, it will be appreciated that the invention can assume many alternative configurations without departing from the core principles of tactile feedback and remote operation. For example, in some embodiments the cobot may include a different number of robotic arms, a single-arm system or even more than two arms. A single-arm variant might be used for simpler interventions or in combination with human assistants, whereas a multi-arm system could handle more complex tasks (e.g., two arms operating and a third arm handling a camera or retractor). Each arm in these variants would still incorporate the tactile sensor arrays and be controlled through the same feedback and safety architecture described.

In other embodiments, the robot need not have a humanoid appearance at all. The system could be implemented as a robotic surgical cart or tower with one or more arms attached. In a non-humanoid variant, the base might be wheeled or track-mounted for repositioning in the OR, but once in place, only the arms (which could be slim and purely functional in design) extend into the operative field. FIG. 10 for instance could equally represent a cart-based robot positioned at the patient’s side. Another variant is an overhead-mounted surgical manipulator: the robotic arms 110 could be mounted on a ceiling gantry or an overhead rail system in the operating room. In that configuration, the arms descend from above the patient. This has the advantage of keeping the floor area clear and potentially enlarging the workspace around the operating table. Each arm, whether mounted on the ceiling or a wall, still contains the same sensor suite 140 and is governed by the same control and feedback systems (150, 160, 170, etc.) as described. The overhead variant would also integrate with the sterile field by deploying sterile draping on the portions of the arms that come down into the field, much like surgical lights and other overhead equipment are managed.

Additionally, the cobot might be a modular add-on to existing surgical robot platforms. For instance, the tactile feedback and communication subsystems could be retrofitted onto a traditional surgical robot (such as a laparoscopic robot) to enable remote operation with haptic feedback. In such cases, the mechanical configuration might differ (maybe the robot arms are laparoscopic instrument manipulators rather than free-moving anthropomorphic arms), but the principles of multi-degree-of-freedom sensing and force reflection remain the same.

The system’s design allows for modularity and tool interchangeability across these variants. The end-effector 115 of each arm can be configured with quick-change couplings for different surgical instruments or sensors. One procedure might call for an endoscopic camera on one arm and a gripper on the other; another might use a scalpel tool on one arm and a suction tool on the other. The control system 150 can recognize what tools are attached (through tool ID chips or calibration data) and adjust the control/feedback parameters accordingly (for example, knowing the different weight or force characteristics of each tool). In an overhead gantry embodiment, tools could even be changed via a tool carousel or exchange system integrated into the ceiling unit, with minimal human intervention.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A perioperative cobotic system for telesurgical interventions with real-time tactile and haptic feedback, the system comprising:
a humanoid cobot platform located at a patient site;
one or more robotic arms on the cobot platform, each robotic arm configured for sub-millimeter precision in manipulating surgical instruments;
a multi-degree-of-freedom tactile sensor array integrated into an end effector of one of the robotic arms, the tactile sensor array being configured to detect force, pressure, slip, and surface features during a surgical interaction;
a haptic feedback system configured to deliver reconstructed tactile sensations to a remote surgeon via a haptic interface device; and
a bi-directional communication architecture operatively connecting the cobot platform to a remote surgical control console, the communication architecture being configured to support real-time control of the robotic arm and real-time haptic feedback with minimized latency and including prediction buffering to compensate for communication delays.

2. The system of claim 1, wherein the tactile sensor array comprises a plurality of sensor elements disposed on a contact surface of the end effector and is configured to provide multi-axis measurements of force, pressure, incipient slip, and surface features encountered during use of the surgical instrument.

3. The system of claim 1, wherein each robotic arm comprises at least seven degrees of freedom and includes high-resolution actuators and sensors to achieve the sub-millimeter precision in positioning the surgical instrument.

4. The system of claim 1, wherein the haptic feedback system comprises a wearable force-feedback glove or an exoskeletal controller, the device being configured to convey to the remote surgeon tactile sensations corresponding to the force, pressure, slip, and surface features detected by the tactile sensor array.

5. The system of claim 1, wherein the bi-directional communication architecture comprises a network interface configured to maintain end-to-end latency below a predefined threshold and a predictive buffering module that anticipates control inputs and tactile feedback signals to mitigate effects of communication latency, thereby enabling substantially real-time teleoperative control.

6. The system of claim 1, wherein the system further comprises a context-aware feedback module configured to adjust at least one of a feedback frequency or a feedback sensitivity of the haptic feedback based on a context of a surgical procedure, the context including at least one of a current phase of the procedure, an anatomical region of the patient, or a type of surgical instrument being used.

7. The system of claim 1, wherein the system includes a safety compliance subsystem configured to monitor forces applied by the robotic arm and to automatically prevent the robotic arm from exerting forces beyond a predetermined threshold or to halt movement of the robotic arm upon detecting an anomalous condition indicative of unintended contact with tissue.

8. The system of claim 1, wherein the system further comprises a logging module configured to record, in real time, data from the surgical procedure including tactile sensor data, control inputs from the surgeon, and video feed, the logging module tagging the recorded data with timestamps and metadata for use in surgical training or audit.

9. The system of claim 1, wherein the cobot platform is configured to maintain sterile field compliance by including surfaces that are sterilizable or drapable with sterile covers, thereby allowing operation of the cobot platform within a sterile surgical environment.

10. The system of claim 1, wherein the system is deployable in either a remote teleoperated mode or a local mode, such that the surgical procedure can be performed by a surgeon from a remote location via the communication architecture or by a surgeon co-located with the patient using a local control interface.

11. A surgical control console for teleoperatively operating a cobotic surgical system with tactile and haptic feedback, the console comprising:
an operator control interface including at least one force-feedback haptic device operable by a surgeon, the haptic device being configured to capture control inputs from the surgeon and to provide haptic feedback to the surgeon;
a visualization display system configured to present an immersive view of a surgical site to the surgeon;
a communication interface configured to transmit control signals to and receive tactile sensor data from the cobotic surgical system in real time; and
a processing unit configured to translate the control inputs into commands for the cobotic surgical system, process tactile sensor data received from the cobotic surgical system into haptic feedback signals for the operator control interface, and provide user-adjustable control parameters including a force scaling factor and a tactile feedback filtering setting.

12. The console of claim 11, wherein the visualization display system comprises a stereoscopic display or a head-mounted display configured to provide a three-dimensional, high-definition visualization of the surgical site based on real-time images captured by one or more cameras associated with the cobotic surgical system.

13. The console of claim 11, wherein the processing unit is configured to allow the surgeon to adjust the force scaling factor to amplify or attenuate transmitted forces and to adjust the tactile feedback filtering setting to filter out noise or irrelevant tactile information.

14. The console of claim 11, wherein the operator control interface comprises a wearable force-feedback glove or an exoskeletal controller worn by the surgeon, the interface being configured to detect movements of the surgeon’s hand or arm and to apply corresponding resistive forces to convey tactile sensations back to the surgeon.

15. The console of claim 11, wherein the console further comprises an emergency stop control accessible to the surgeon, the emergency stop control being configured to immediately halt motion of the robotic arms of the cobotic surgical system upon activation.

16. The console of claim 11, wherein the processing unit is configured to execute predictive control algorithms that anticipate the surgeon’s control inputs and buffer command data to compensate for communication latency between the console and the cobotic surgical system.

17. A method of performing a remote surgical procedure using a teleoperatively controlled cobotic surgical system, the method comprising:
receiving, from a remote surgical control console operated by a surgeon, control commands to manipulate a patient-side robotic arm;
manipulating, in response to the control commands, at least one robotic arm of a humanoid cobot platform at a patient site to operate a surgical instrument with sub-millimeter precision;
detecting tactile data via a multi-degree-of-freedom tactile sensor array integrated in an end effector of the robotic arm, the tactile data including measurements of force, pressure, slip, and surface features encountered during the surgical instrument’s interaction with patient tissue;
transmitting the tactile data from the cobot platform to the remote surgical control console over a bi-directional communication link that minimizes latency, the communication link employing predictive buffering to maintain real-time feedback;
generating haptic feedback signals at the remote surgical control console based on the tactile data;
delivering the haptic feedback signals to a force-feedback haptic interface device at the remote surgical control console to provide the surgeon with tactile sensations corresponding to the conditions at the patient site;
adjusting at least one of a feedback frequency or a feedback sensitivity of the haptic feedback signals based on a context of the surgical procedure, the context including at least one of a surgical phase, an anatomical region, or a tool type involved in the procedure;
applying safety compliance algorithms by monitoring forces on the surgical instrument and automatically preventing the robotic arm from exceeding a safe force threshold or halting movement of the robotic arm upon detecting an anomalous contact condition; and
logging, in real time, procedural data including the tactile data, the control commands, and video of the surgical site, wherein the procedural data are time-synchronized and stored for subsequent training or audit.

18. The method of claim 17, wherein the remote surgical control console comprises a master haptic controller, and receiving the control commands includes capturing the surgeon’s input motions via the master haptic controller while delivering the haptic feedback signals includes actuating the master haptic controller to apply corresponding tactile forces to the surgeon.

19. The method of claim 17, further comprising providing the surgeon with an immersive visual display of the surgical site by streaming a real-time stereoscopic video feed from the cobot platform to the remote surgical control console during the surgical procedure.

20. The method of claim 17, wherein the humanoid cobot platform at the patient site is maintained within a sterile field during the surgical procedure by use of sterile drapes or sterilizable coverings, while the remote surgical control console is located outside the sterile field.
